(19) United States
(12) Patent Application Publication
(10) Pub. No.: US 2012/0016264 A1
(43) Pub. Date: Jan. 19, 2012

(54) Microextraction Probe

(76) Inventors: Jeff Weidenhamer, Ashland, OH (US); Brian Mohney, Ashland, OH (US)

(21) Appl. No.: 12/835,515

(22) Filed: Jul. 13, 2010

(51) Int. Cl. A61B 5/15; G01N 35/10

(52) U.S. Cl. 600/576; 73/864.21

(57) ABSTRACT

The instant invention is directed to devices for the collection of chemical substances from substantially undisturbed natural environs for subsequent identification and quantification.

### GOVERNMENT INTEREST

The research associated with this invention was funded in part by NSF Grant No. DEB 0515826. Accordingly, the United States Government may have certain rights with regard to in this invention.

### FIELD

This invention relates to the field of mechanical devices for in situ collection of chemical substances from substantially undisturbed natural environs for subsequent isolation, identification and quantification.

### BACKGROUND

Current methods for field testing for chemicals from various sources such as soil, air, water, blood, fruit juice from growing fruit and other solids and fluids generally require substantial disruption of the test area. For example, where the test site comprises soil, digging and removing a sample of the soil for further work-up in the laboratory, e.g., isolation, identification and quantification of chemical compounds present in the soil is the usual procedure. Similarly, where the test sites comprises a fluid, collecting and isolating a sample of the fluid from its source for further work-up in the laboratory is the norm. It would be of considerable benefit to be able to collect the sought—after compounds directly from the natural environs both for single pass analysis and, perhaps more significantly, for longer term continuous or intermittent studies of the presence and amount of the subject compounds over time. An exemplary non-limiting example is the collection of soil samples for the study of the dynamics of environmental contamination or for the study or allelopathic interactions among plants growing in the soil.

As mentioned above, field testing for chemical compounds in soil generally involves removal of samples by digging or core-drilling into the soil, removing a volume of soil and then examining the removed soil samples under laboratory conditions for the presence of the sought after compounds. To perform further tests to determine whether the amount of the compounds found is increasing, decreasing or remaining constant requires re-digging or re-drilling to obtain additional samples for testing. It would be of substantial value to be able to collect compounds from relatively undisturbed soil and to be able to do continuously or at intervals over relatively long periods of time without having to disturb the natural environment of the test site each time a sample is collected. For instance, not only would this facilitate the study of soil contamination, the ability to collect chemicals from undisturbed soil over a long period of time would also benefit the study of the natural dynamics and mechanisms of naturally-occurring in-soil biochemical processes. For example, without limitation, the study of allelopathy among plants would be greatly enhanced by such capability.

Allelopathy is the production and dissemination into the environment of chemicals by organisms such as plants, algae, bacteria and fungi, which chemicals influence the growth, survival and reproduction of other organisms in the vicinity of the chemical-producing plant. Allelopathy can be positive, that is can have a beneficial effect on target organisms or it can be negative and have a negative effect on target organisms. At present, the term is most commonly applied to the negative effects of biochemicals produced by certain plants on survival of organisms in the vicinity of the producing plant.

The problem with the study of allelopathy is that many experiments are conducted in the laboratory under artificial conditions where it is difficult to reproduce natural conditions and/or to relate the result to that which occurs in an ecological environment. Field studies, on the other hand, are fraught with problems because the generally disruptive nature of experimental procedures that tend to introduce variables that may be difficult to account for. The device and methods of the instant invention will provide tools which will substantially advance the study and understanding of ecological phenomena such as allelopathy as well as many other ecological conditions having chemical aspects such as the study of the dynamics of environmental contamination of soil and water.

### SUMMARY

Thus, an aspect of the instant invention is a device, comprising:

an elongate hollow core having a proximal end, a distal end and a lumen extending from the proximal to distal end;
an elongate diffusion permeable tubing having a proximal end and a distal end, wherein:
the distal end of the tubing enters the lumen at or near the core's proximal end;
the distal end of the tubing threads through the lumen to a point at or near the distal end of the core;
the distal end of the tubing exits the lumen through a through-hole in a wall of the core;
the tubing is helically wound around the core from the exit point back toward to proximal end of the core for a predetermined distance;
upon achieving the predetermined distance the distal end of the tubing reenters the lumen through a second through-hole;
the distal end tubing then threads back through the core to a point at or near the proximal end of the core; and
the distal end of the tubing exits the lumen;
   a retractable/removable housing enclosing the core with helically-wound tubing from its distal end to a point near, at or beyond the proximal end of the core; and
   a penetrating tip, having a proximal end and a distal end, the proximal end being permanently or detachably coupled to the distal end of the elongate core and detachably coupled to the distal end of the housing.

In an aspect of the invention, the elongate core comprises a metal, a polymer, a ceramic or any combination thereof.

In an aspect of this invention, the elongate core has a length of about 1 cm to about 100 cm.

In an aspect of the invention, the elongate core is cylindrical in shape.

In an aspect of the invention, the elongate core has a cross-sectional diameter of about 3 mm to about 7 mm.

In an aspect of the invention, the diffusion permeable tubing has a wall thickness of about 0.1 to about 1.0 mm.

In an aspect of the invention, the diffusion permeable tubing has a length of about 10 cm to about 20 meters.

In an aspect of the invention, the diffusion permeable tubing comprises a polymer.

In an aspect of the invention, the polymer comprises a silicone polymer.

In an aspect of the invention, the silicone polymer comprises poly(dimethylsiloxane).

In an aspect of the invention, some or all the cross-sectional dimensions of the penetrating tip decrease from its proximal end to its distal end.

In an aspect of the invention, the penetrating tip is substantially conical and substantially pointed at its distal end.

In an aspect of the invention, the penetrating tip comprises a recessed space on an inner surface of its proximal end, the space having substantially the same cross-sectional shape as the distal end of the housing such that the housing fits into the recessed space.

In an aspect of the invention, the cross-sectional shape of the housing is substantially cylindrical.

In an aspect of the invention, the cross-sectional diameter of the housing is about 6 mm to about 10 mm.

In an aspect of the invention, a fluid injector is detachably coupled to the proximal end of the diffusion permeable tubing.

In an aspect of the invention, the fluid injector comprises a syringe.

In an aspect of the invention, a sample collector is detachably coupled to the distal end of the diffusion permeable tubing.

In an aspect of the invention, the housing is constructed of a metal, a polymer, a ceramic or any combination thereof.

In an aspect of the invention, the penetrating tip is constructed of a metal, a polymer, a ceramic or any combination thereof.

In an aspect of the invention the housing and the penetrating tip are constructed of a metal.

In an aspect of the invention, the housing and the penetrating tip are constructed of a polymer.

An aspect of the instant invention is a method of obtaining a sample from a test site, comprising:

identifying a test site to be sampled;
advancing the penetrating tip of the device of claim 1 until the helically wound diffusion permeable tubing is substantially adjacent to the test area;
retracting/removing the housing leaving the helically-wound tubing in contact with the environment at the test site;
at selected intervals injecting a fluid into the proximal end of the helically-wound tubing;
advancing the fluid to a region of the tubing where an outer wall of the tubing is in contact with the test site;
allowing the fluid to remain in contact with an inner wall of the helically-wound tubing opposite the outer wall of the tubing that is in contact with the test site for a selected time period;
ejecting the fluid from the distal end of the helically-wound tubing into a collection vessel;
optionally repeating, as many times as desired, injecting a fluid, which for each repetition may be the same as or different from any previously injected fluid, at selected intervals, which may be the same as or different from any prior interval(s), maintaining contact of the fluid with the tubing wall for a selected time, which may be the same as or different than any previous contact time, and ejecting each interval sample individually into a different collection vessel or ejecting some or all of the interval samples into the same collection vessel.

In an aspect of this invention, in the above method, the fluid comprises a solvent.

In an aspect of this invention, in the above method, the solvent comprises an organic solvent.

In an aspect of this invention, in the above method, the fluid comprises an acidic or basic buffer.

In an aspect of this invention, in the above method, the test site comprises a test solid.

In an aspect of this invention, in the above method, the test solid comprises soil.

In an aspect of this invention, in the above method, the soil is suspected of containing compounds indicative of allelopathic activity among plants in the vicinity of the test site.

In an aspect of this invention, in the above method, the soil is suspected of being contaminated.

In an aspect of this invention, in the above method, the test site comprises a test fluid.

In an aspect of this invention, in the above method, the test fluid comprises ground water suspected of being contaminated.

In an aspect of this invention, the test fluid comprises a body of water; a beverage; fruit juice in intact growing fruit; a food product in its raw state, its processed ready-to-package state or its ready-to-eat state; blood in an organ or blood vessel of a living animal; or sap in a living plant.

### BRIEF DESCRIPTION OF THE FIGURES

FIG. 1 is a schematic depiction of a probe of this invention.

FIG. 2 is a schematic depiction of a core of the present invention with through-holes.

### DISCUSSION

It is understood that with regard to this description and the appended claims, any reference to any aspect of this invention made in the singular includes the plural and vice versa unless it is expressly stated or unambiguously clear from the context that such is not intended.

As used herein, any term of approximation such as, without limitation, near, about, approximately, substantially, essentially and the like mean that the word or phrase modified by the term of approximation need not be exactly that which is written but may vary from that written description to some extent. The extent to which the description may vary will depend on how great a change can be instituted and have one of ordinary skill in the art recognize the modified version as still having the properties, characteristics and capabilities of the modified word or phrase. In general, but with the preceding discussion in mind, a numerical value herein that is modified by a word of approximation may vary from the stated value by ±15%, unless expressly stated otherwise.

As used herein, “optional” means that the element modified by the term may or may not be present.

As used herein, the terms “preferred,” “preferably,” and the like refer to the situation as it existed at the time of filing this patent application.

As used herein, the terms “proximal” and “distal” do not have absolute meaning; rather they are used to define relative positions of the various elements of the device of this invention. Thus, the description of the core element of this invention as having a proximal and a distal end simply refers to the two ends of the core and until some other element of the device is described relative to those proximal and distal ends, either end of the core may be considered the proximal end or the distal end.

It is noted that there exists a marginally related sampling technique to that of this invention, solid phase microextraction (SPME). There is, however, a significant difference between SPME and the current device and technique. SPME is a solventless procedure while the present invention comprises a solvent extraction step for obtaining the compounds from the diffusion permeable tubing. The solvent extraction technique of the present invention is particularly suitable for samples for which HPLC analysis is contemplated and there are a number of important classes of compounds for which HPLC is in fact the preferred analytical tool. Thus, the differences between the tools and procedure of the two techniques overwhelm any perceived similarities between them. Clearly, they each have their particular use domains, which, while they may overlap at times, are distinctly different at most times and in most applications.

As used herein, a “test site” simply refers to a region to be accessed by a probe of this invention for the collection of chemical compounds to be identified and, if desired, quantified. A test site may, for example, without limitation, comprise a “test solid” such as, without limitation, soil beginning at a particular depth below ground level and extending for a selected distance further into the soil. A solid test site may also comprise an animal or plant tissue in which biochemical processes are being studied or in which the present and amount of toxins is being investigated. The test site may also comprise a “test fluid” such as air, ground water or a body of open water, i.e., a stream, river, lake, ocean, sea, gulf and the like, at a particular depth. The test site may comprise a test fluid such as juice in an intact fruit growing in its natural environment. The test site may comprise a beverage such as, without limitation, wine, beer, gin, scotch, vodka, soft drink, fruit drink or any other potable liquid to be tested for the presence of trace contaminants or in which analysis of the components contributing to the beverage's unique flavor characteristics is being conducted. The test site may also comprise virtually any food product, either in its raw unprocessed state where, for example without limitation, the food product is being continuously or at intervals examined for the presence of undesirable chemical compounds, which may comprise, among others, toxins or chemicals that, while not directly harmful to health, affect the taste and/or appearance of the food product. The food product may also be in its processed, ready to package or even further down-stream in its ready-to-eat state where continuous or interval testing is performed to monitor the product. A test site test fluid may comprise blood within an organ or the blood stream of a living animal. Similarly, the test site may comprise a test fluid such as the sap of a growing plant. Many other test sites comprising test solids and/or test fluids that will be amenable to testing using devices of this invention will become apparent to the skilled artisan based on the disclosures herein and any and all such test sites, test solids and test fluids are within the scope of this invention.

As used herein an “elongate hollow core” simply refers to a length of material having a lumen that traverses its entire length. In a presently preferred embodiment, the elongate hollow core simply comprises a pipe-like structure, as such are commonly known to skilled artisans and laypersons alike. Thus, the elongate core may, and preferably at present does, have a cylindrical shape with a circular cross-section as such is most usually associated with pipes, but it also may have any other cross-sectional shapes such as, without limitation, square, triangular, elliptical, etc. The core is constructed of an essentially inflexible material such as metal, ceramic, glass, polymer, etc. Presently preferred are metals such as, without limitation, stainless steel, aluminum, iron, copper, galvanized metals, etc. and polymers such as, without limitation, polyethylene, polypropylene, polyvinyl chloride, poly(tetrafluoroethylene) (Teflon®) and the like. Other materials may be used to fabricate the core so long as they exhibit the requisite properties of sufficient strength, rigidity and inertness. With regard to inertness, the material of which the core is fabricated must not in any manner compete with the diffusion permeable tubing by absorbing or complexing with the chemical substance or substances that are the object of the particular study.

Preferably at present, the hollow core has an outside diameter of about 3 mm to about 7 mm although, depending on parameters such as, without limitation, the environs where sampling is to occur, the size of the test area and the length of diffusion permeable tubing that is to be exposed to the test area and the type of study being conducted, the hollow core may be substantially larger in diameter. Likewise, the length of the core may vary substantially but at present a length of about 1 cm to about 100 cm is contemplated. Those skilled in the art will be able to readily determine the desired core length and diameter for a particular use with very little if any experimentation based on the disclosure herein.

As used herein, “elongate diffusion permeable tubing” refers to a flexible tube having, preferably at present, a cylindrical shape of essentially constant outside diameter and circular cross-section with a lumen extending from its proximal to its distal end. “Diffusion permeable” refers to the property of the material of which the tubing is fabricated by which molecular substances in contact with the outer surface of the tubing, that is, the surface in direct contact with the test site environment, traverse the wall thickness of the tubing from the outer wall to the inner wall that defines the lumen of the tubing. The wall of the diffusion permeable tubing should be a thin as possible without compromising the integrity of the tubing so as to permit efficient and relatively rapid movement of molecular materials in contact with the outer wall of the tubing to its inner wall. It is presently preferred that tubing having a wall thickness of about 0.1 mm to about 1.0 mm be used. The length of tubing to be exposed to a test site may vary substantially depending on the substances to be sampled, the size of the test site, etc. but at present a tubing length of about 10 cm to about 20 meters is contemplated.

While any material that has the requisite strength when thin, is flexible and is permeable to molecular substances may be used as the diffusion permeable tubing, polymeric materials, in particular silicon-based polymers such as, without limitation, polydimethylsiloxane (PDMS), are presently preferred. PDMS is particularly preferred at present because its diffusion permeability to chemical compounds has been demonstrated in various studies. That is, PDMS has been used in experiments designed to study the physical characteristics of chemical compounds in water: Ooki, A. and Yokouchi, Y., Development of a Silicon Membrane Tube Equilibrator for Measuring Partial Pressures of Volatile Organic Compounds in Natural Water, Environmental Science and Technology, 2008, 42(15):5706-5711; and in tissues and lipids, Mayer, P. et al., Silicone Membrane Equilibrator: Measuring Chemical Activity of Nonpolar Chemicals with Poly(dimethylsiloxane) Microtubes Immersed Directly in Tissue and Lipids, Analytical Chemistry, 2009, 81(4): 1536-1542. An exemplary, non-limiting PDMS material for use in the fabrication of a device of this invention is Dow Corning Silastic® Laboratory tubing, which is currently available in multiple sizes including without limitation at least two from Fisher Scientific, Catalog Nos. 508-003 (0.64 mm ID×1.19 mm OD) and 508-001 (0.3 mm ID×0.64 mm OD).

In the fabrication of a probe of this invention, the diffusion permeable tubing passes through the lumen of the core to a point at or near the distal end of the core where it exits the lumen by means of a through-hole in the wall of the core. The proximal end of the tubing is maintained at the proximal end of the core where it is accessible for coupling to solvent and gas delivery devices. The through-hole may be sized so that the tubing fits loosely through it. Alternatively, the through-hole may be sized so as to form a relatively tight (preferably at least water-tight) fit with the tubing. In yet another embodiment, the through-hole may be sized somewhat larger than the OD of the tubing and comprise a flexible diaphragm made of, without limitation, materials such as latex rubber or silicone elastomer that has a small hole in it that can expand and conform to the shape and size of the tubing to allow for the use of various size tubing with the same core. The core may have a plurality of such through-holes along its length to permit various lengths of helically-wound diffusion permeable tubing to be exposed to the environment at the test site.

After exiting the lumen of the core, the tubing is helically wound around the core back toward the proximal end of the core for a predetermined distance based on the length of the test region to be sampled. The tubing then re-enters the lumen of the core by means of another through-hole similar to the one described previously and then threads back though the lumen to the proximal end of the tubing where it exits the lumen. Both ends of the tubing are thus accessible at the proximal end of the core for coupling with fluid and gas delivery devices, sample collection devices and analytical devices such as, without limitation, gas chromatographs, liquid chromatographs and the like.

A penetrating tip is located at the distal end of the core. Its proximal end may be permanently coupled to the distal end of the core or it may be fitted to the diameter of the core in such a manner that, after advancement of the core to a test site and the conduction of desired experiments, the core may be withdrawn from the test site leaving the penetrating tip behind. The penetrating tip can have any shape that is conducive to facilitating insertion of the device through whatever material is encountered at a particular test site. For example, without limitation, it is contemplated that the device of this invention will be particularly useful for collection of chemical compounds from native soils so the penetrating tip must be capable of penetrating into soils that may be relatively soft such as sand, barns and the like or may be relatively dense and difficult to penetrate such as packed clays, gravel, etc. Thus, the penetrating tip may be conical with a circular base at its proximal end which is coupled to the distal end of the core and a substantially pointed apex at its distal end, it may be pyramid-shaped, it may be spade-shaped, it may be arrow-head shaped, etc. Presently preferred is a conical shape. The penetrating tip is constructed of a material strong enough to withstand forces exerted on it during the penetration process. Presently preferred are metallic materials such as iron, stainless steel, aluminum, copper alloys and the like, although tough, hard plastics such as, without limitation, high density polyethylene, polyamides (nylons), and the like may also be suitable.

The core with diffusion permeable tubing helically wound around it is enclosed in a housing that protects the tubing during delivery of the device to a test site. The housing is fabricated of a material that has essentially the same physical properties as the penetrating tip; i.e., it must be strong enough to withstand the rigors of being advanced to a particular test site. For example, the housing may be fabricated of a metal, such as without limitation, stainless steel, aluminum or iron or it may be fabricated of a polymer of sufficient strength such as polyvinyl chloride, polyethylene, polypropylene and the like. The housing is sized so as to have an ID that is greater than the diameter of the core plus the helically-wound tubing. The distal end of the housing is fitted to the proximal end of the penetrating tip so as to form a smooth transition from the OD of the housing to the tapered conical penetrating tip. In the alternative, the housing may have an OD that is somewhat smaller than the diameter of the proximal end of the penetrating tip. In either case, the desired result is that the housing does not interfere with the advancement of the device into a test site. Once the test site is reached, the housing is retracted to expose the diffusion permeable tubing to the test site environs.

The proximal end of the housing may be approximately even with or it may extend beyond the proximal end of the core. In the latter instance, it is contemplated that the proximal and distal ends of the diffusion permeable tubing could be folded into the extended portion of the housing so as to permit tapping the housing with a hammer or other device to facilitate penetration of the device into a test site without harming the tubing.

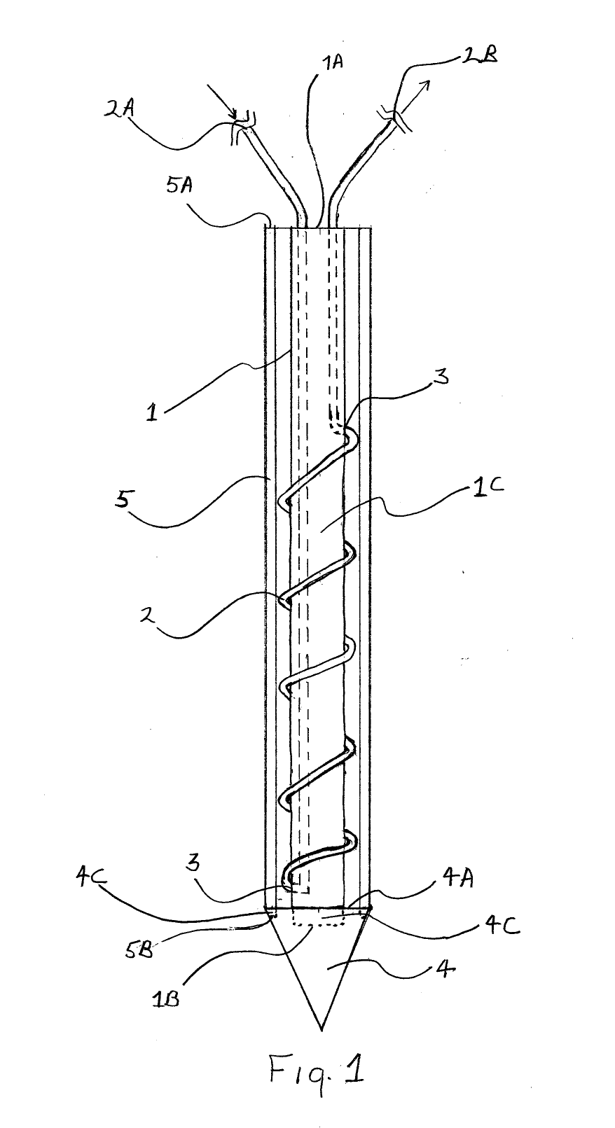

A schematic of a device of this invention is shown in FIG. 1. It is understood that the schematic is provided for the edification of the reader only and is not intended to be in any manner limiting on the scope of this invention. That is, changes may be made in various aspects of the device as shown in FIG. 1 without materially affecting the overall concept of the device and its use and all such changes are within the scope of this invention.

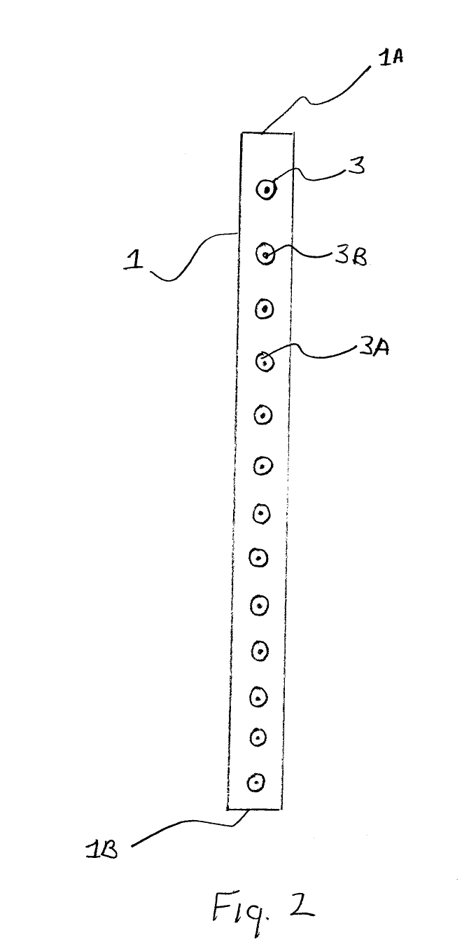

Elongate hollow core 1 has proximal end, 1A, distal end 1B and lumen 1C. As was mentioned previously, core 1 can have any cross-sectional shape such as, without limitation, circular, triangular, square, pentagonal, etc. It is, however, presently preferred that the cross-section be substantially circular, that is, that the elongate hollow core be what is commonly referred to by both skilled and lay persons as a “pipe.” The proximal end is generally open but may be capped such as, without limitation, a plastic lid. The plastic lid may include through-holes having a diameter that will permit the passage of permeable tubing 2 into lumen 1C. Core 1 has through-holes 3 in its wall at intervals along its length as shown in FIG. 2. Through-holes 3 are, in one embodiment, sized to have a diameter that is marginally larger than the OD of the permeable tubing such that the tubing fits easily through the through-hole and does not form any type of seal. In an alternative embodiment the through-hole has a diameter that is only very slightly larger than the diameter of the diffusion tubing to result in a snug fit, i.e., at least water-tight, when diffusion permeable tubing 2 passes through them. In yet another embodiment, through-holes 3 may have a nominally larger diameter than the tubing, the through-holes 3 being sealed with a diaphragm 3A that has a small expandable aperture 3B that can expand and conform to the diameter of the diffusion tubing being passed through.

Proximal end 4A of penetrating tip 4 is permanently or detachably coupled to the distal end 1B of hollow core 1 by fitting distal end 1B or core 1 into recess 4C of penetrating tip 4. Penetrating tip 4 has any shape that will facilitate the advancement of the device thought the medium is being tested. For example, without limitation, if the device of this invention is being used to test for chemicals in soil, the penetrating tip might have a conical shape as shown in FIG. 1. If desired, the penetrating tip may have any other shape that will facilitate the passage of the device through whatever materials the test region is located. Thus, shapes such as spade, arrow tip, tapered cross, etc., may be used. If the device is being used to test tissues, the tip may consist essentially of a tapered needle-like structure to permit more gentle and less destructive intrusion into the tissue.

Diffusion permeable tubing 2 passes through lumen 1C from core proximal end 1A and exits the core at or near distal end 1B through one of through-holes 3. Tubing 2 is then helically wound around core 1 from the point of exit from lumen 1C back toward proximal end 1A of core 1. When a predetermined length of tubing 2 has been helically wound around core 1, tubing 2 reenters core lumen 1C through another of through-holes 3. It is understood that, while tubing 2 is shown in FIG. 1 as being loosely helically wound around the core such that there is a substantial distance between the helical turns and it is further understood that such a configuration is entirely possible and clearly within the scope of this invention, the spacing of the helical turns can be, and generally is, much closer together to the point where outer wall of tubing 2 is continuously in contact over the entire span of the helical wind. When diffusion permeable tubing 2 has been fully deployed, both proximal end 2A and distal end 2B of tubing 2 are exposed as accessible at proximal end 1A of core 1.

Housing 5 has proximal end 5A and distal end 5B and a length that causes it to fully enclose helically-wound core 1 from distal end 1B of core 1 to proximal end 1A of core 1 or, optionally, beyond as discussed above. The diameter of distal end 5B of housing 5 is slightly smaller than the diameter of proximal end 4A of penetrating tip 4 such that housing 5 fits into recess 4C of penetrating tip 4. In FIG. 1, proximal end 5A of housing 5 is shown as being essentially even with proximal end 1A of core 1. As has been mentioned previously, however, proximal end 5A of housing 5 may extend beyond proximal end of core 2 for a sufficient distance to be capable of harboring the proximal and distal ends of diffusion tubing 3 and protect them from damage is a device such as a hammer is used to drive the device into a material such as soil to a designated test site.

An exemplary manner for employing the probe of this invention, where the test site is at a particular depth below the surface of soil is as follows:

Once the test site and test site length has been determined, the probe is assembled by inserting the proximal end of the diffusion permeable tubing through a selected through-hole at the distal end of the core and threading the tubing through the lumen to the proximal end of the core where it is accessible for attachment to solvent delivery devices such as, without limitation, a syringe or peristaltic pump. The diffusion tubing is then helically wound around the core from the point of exit from the lumen through the through-hole back toward the proximal end of the core until the desired length of tubing and of helical wind is achieved at which time the distal end of the tubing is passed through another through-hole and then threaded through the core lumen back to the proximal end of the core where it becomes accessible for coupling to sample collection devices or analytical tools. The distal end of the core is then inserted into the penetrating tip if the tip is separate from the core. The housing is then slipped down over the core and helically-wound tubing and engages the proximal end of the penetrating tip. The device is then ready to be advanced to the selected test site either by hand pressure if the soil is loose enough or by first protecting the diffusion tubing by folding it into the proximal end of the housing where the proximal end extends beyond the proximal end of the core and then tapping on the housing with a hammer to advance the device to the test site. If the test site is particularly deep, a core-drill may be used to approach the test site and then the probe is advanced the final distance by exerting pressure on the proximal end of the housing. The proximal and distal ends of the diffusion tubing are then recovered from the housing and rendered accessible for attachment of solvent delivery devices and sample collectors.

Once the probe is in position, the housing is removed to expose the diffusion tubing to the environment that comprises the test site. After the diffusion tubing has been in contact with the test site for a predetermined period of time, a bolus of solvent is passed through the tubing by means of a syringe, peristaltic pump or other controllable delivery means. The volume of the solvent bolus and the speed with which it is passed through the portion of the diffusion tubing that is in contact with the test site is empirically determined. The bolus of solvent is then pushed through the diffusion permeable tubing to its distal end where it is deposited in a collection vessel which may be a flask, a test tube, a vial or any other manner of collection device. The foregoing procedure can be repeated at selected intervals for as long as desired, the solvent from each pass being combined with other samples in the collection vessel at the distal end of the tubing or each sample being individually collected. The samples may then be passed without further manipulation into a analytical device such as, without limitation, a gas chromatograph (GC), a high performance liquid chromatograph (HPLC), a mass spectrometer (MS), an infrared spectrometer, a UV-visible light spectrometer or any other analytical device useful for the detection and identification of organic compounds and any combination of such devices such as a GC-MS or the like.

The choice of solvent to be passed though the diffusion permeable tubing depends to a great extent on the material of which the tubing is fabricated. For example, if PDMS tubing is used, a viable solvent choice is a lower alcohol, in particular methanol. This is so because methanol and presumably other lower alcohols are not excessively absorbed by PDMS and therefore the diffusion characteristics of the PDMS will not be deleteriously affected by the solvent. That is, it has been shown that PDMS absorbs only about 1.5% wt/wt methanol whereas it absorbs about 5.5% wt/wt of 1-octanol, 129% wt/wt pentane and 136% wt/wt toluene at equilibrium. If a different diffusion permeable tubing material is used, determination of proper solvents is a relatively easy matter, the simplest approach being to soak the tubing in a volume of various solvents and then measure the difference in weight of the tubing before and after the immersion. Of course, the solvent choice will also have to take into consideration the nature of the chemical compound(s) being tested for. For example, without limitation, it may be that ethanol, while slightly more absorbable by PDMS is a much better solvent for the target compound(s) and therefore a better overall choice of solvent. The skilled artisan will be able to readily determine the best solvent based on the disclosure herein.

It has been found, and it is a presently preferred embodiment of this invention, that aqueous acidic or basic buffers can be used as the solvent for collecting samples from the inner walls of diffusion permeable tubing including PDMS. The use of such solvents permits the trapping of ionizable acids and bases such as substituted benzoic and phenolic acids.

It is also an embodiment of this invention that a derivatization reagent be incorporated into the polymeric matrix of the diffusion permeable tubing such that, as compounds diffuse through the tubing they, or least those that are reactive toward the derivatization reagent, react and form a reaction product that might be, without limitation, more stable, more volatile, less volatile, more soluble in the elution solvent and/or simply more suitable for analysis by the selected analytical technique. The derivatized compound is then eluted from the tubing by the solvent and carried to the sample collector.

What is claimed:

1. A device, comprising:
an elongate hollow core having a proximal end, a distal end and a lumen extending from the proximal to distal end;
an elongate diffusion permeable tubing having a proximal end and a distal end, wherein:
the distal end of the tubing enters the lumen at or near the core's proximal end;
the distal end of the tubing threads through the lumen to a point at or near the distal end of the core;
the distal end of the tubing exits the lumen though a through-hole in a wall of the core;
the tubing is helically wound around the core from the exit point back toward to proximal end of the core for a predetermined distance;
upon achieving the predetermined distance the distal end of the tubing reenters the lumen through a second through-hole;
the distal end tubing then threads back through the core to a point at or near the proximal end of the core; and
the distal end of the tubing exits the lumen;
a retractable/removable housing enclosing the core with helically-wound tubing from its distal end to a point near, at or beyond the proximal end of the core; and
a penetrating tip, having a proximal end and a distal end, the proximal end being permanently or detachably coupled to the distal end of the elongate core and detachably coupled to the distal end of the housing.

2. The device of claim 1, wherein the elongate core comprises a metal, a polymer, a ceramic or any combination thereof.

3. The device of claim 2, wherein the elongate core has a length of about 1 cm to about 100 cm.

4. The device of claim 3, wherein the elongate core is cylindrical in shape.

5. The device of claim 4, wherein the elongate core has a cross-sectional diameter of about 3 mm to about 7 mm.

6. The device of claim 1, wherein the diffusion permeable tubing has a wall thickness of about 0.1 to about 1.0 mm.

7. The device of claim 6, wherein the diffusion permeable tubing has a length of about 10 cm to about 20 meters.

8. The device of claim 1, wherein the diffusion permeable tubing comprises a polymer.

9. The device of claim 8, wherein the polymer comprises a silicone polymer.

10. The device of claim 9, wherein the silicone polymer comprises poly(dimethylsiloxane).

11. The device of claim 1, wherein some or all the cross-sectional dimensions of the penetrating tip decrease from its proximal end to its distal end.

12. The device of claim 11, wherein the penetrating tip is substantially conical and substantially pointed at its distal end.

13. The device of claim 1, wherein the penetrating tip comprises a recessed space on an inner surface of its proximal end, the space having substantially the same cross-sectional shape as the distal end of the housing such that the housing fits into the recessed space.

14. The device of claim 13, wherein the cross-sectional shape of the housing is substantially cylindrical.

15. The device of claim 14, wherein the cross-sectional diameter of the housing is about 6 mm to about 10 mm.

16. The device of claim 1, wherein a fluid injector is detachably coupled to the proximal end of the diffusion permeable tubing.

17. The device of claim 13, wherein the fluid injector comprises a syringe.

18. The device of claim 1, wherein a sample collector is detachably coupled to the distal end of the diffusion permeable tubing.

19. The device of claim 1, wherein housing is constructed of a metal, a polymer, a ceramic or any combination thereof.

20. The device of claim 19, wherein the penetrating tip is constructed of a metal, a polymer, a ceramic or any combination thereof.

21. The device of claim 1, wherein the housing and the penetrating tip are constructed of a metal.

22. The device of claim 1, wherein the housing and the penetrating tip are constructed of a polymer.

23. A method of obtaining a sample from a test site, comprising:
identifying a test site to be sampled;
advancing the penetrating tip of the device of claim 1 until the helically wound diffusion permeable tubing is substantially adjacent to the test area;
retracting/removing the housing leaving the helically-wound tubing in contact with the environment at the test site;
at selected intervals injecting a fluid into the proximal end of the helically-wound tubing;
advancing the fluid to a region of the tubing where an outer wall of the tubing is in contact with the test site;
allowing the fluid to remain in contact with an inner wall of the helically-wound tubing opposite the outer wall of the tubing that is in contact with the test site for a selected time period;
ejecting the fluid from the distal end of the helically-wound tubing into a collection vessel;
optionally repeating, as many times as desired, injecting a fluid, which for each repetition may be the same as or different from any previously injected fluid, at selected intervals, which may be the same as or different from any prior interval(s), maintaining contact of the fluid with the tubing wall for a selected time, which may be the same as or different than any previous contact time, and ejecting each interval sample individually into a different collection vessel or ejecting some or all of the interval samples into the same collection vessel.

24. The method of claim 23, wherein the fluid comprises a solvent.

25. The method of claim 24, wherein the solvent comprises an organic solvent.

26. The method of claim 23, wherein the fluid comprises an acidic or basic buffer.

27. The method of claim 23, where the test site comprises a test solid.

28. The method of claim 23, wherein the test solid comprises soil.

29. The method of claim 28, where the soil is suspected of containing compounds indicative of allelopathic activity among plants in the vicinity of the test site.

30. The method of claim 28, wherein the soil is suspected of being contaminated.

31. The method of claim 23, wherein the test site comprises a test fluid

32. The method of claim 31, wherein the test fluid comprises ground water suspected of being contaminated.

33. The method of claim 31, wherein the test fluid comprises a body of water; a beverage; fruit juice in intact growing fruit; a food product in its raw state, its processed ready-to-package state or its ready-to-eat state; blood in an organ or blood vessel of a living animal; or sap in a living plant.
